(19) United States
(12) Patent Application Publication
(10) Pub. No.: US 2020/0390433 A1
(43) Pub. Date: Dec. 17, 2020

(54) RETRACTOR FOR SPINAL SURGERY

(71) Applicant: Solco Biomedical Co., Ltd., Pyeongtaek-si (KR)

(72) Inventors: Hwi Geun Yu, Pyeongtaek-si (KR); Jong Won Hong, Seoul (KR)

(73) Assignee: Solco Biomedical Co., Ltd., Pyeongtaek-si (KR)

(21) Appl. No.: 16/562,970

(22) Filed: Sep. 6, 2019

(30) Foreign Application Priority Data
Jun. 13, 2019 (KR) 10-2019-0069730

(51) Int. Cl. A61B 17/02

(52) U.S. Cl. CPC A61B 17/0206; A61B 2017/0256; A61B 17/025

(57) ABSTRACT

The present invention relates to a retractor for spinal surgery, which allows an operator to obtain a maximum surgical view with a minimal incision by easily widening a skin incision site of a patient while minimizing the surgical incision area.

To this end, the retractor for spinal surgery includes: a first retractor body, which has a semi-cylindrical shape and in which a space is formed to accommodate a plurality of sleeves, and simultaneously, a lower end thereof is inserted into a skin incision site; a second retractor body, which has the same shape as that of the first retractor body and is rotatably coupled to the first retractor body, and thereby accommodate the sleeves along with the first retractor body; and a pair of hinge units, which are mounted on an upper part of the first retractor body and the second retractor body, and in which an end of the first retractor body and an end of the second retractor body are hinged together by a connecting pin.

### CROSS-REFERENCE TO RELATED APPLICATIONS

A claim for priority under 35 U.S.C. § 119 is made to Korean Patent Application No. 10-2019-0069730 filed Jun. 13, 2019, in the Korean Intellectual Property Office, the entire contents of which are hereby incorporated by reference.

### BACKGROUND OF THE INVENTION

### Field of the Invention

The present invention relates to a retractor for spinal surgery, and more specifically, to a retractor for spinal surgery, which is designed to easily widen the skin incision site of a patient while minimizing the skin area for surgery.

### Related Art

Recently, surgery has been performed such that the wound remaining in the skin incision area after surgery can be minimized.

A skin incision site refers to a site where part of the abdomen and part of the vertebra are incised in order to insert a surgical instrument for surgery or to remove the exudates (organs, lumps, cancer tissues, etc.).

However, as the incision area becomes small, it becomes more difficult to secure the space available for surgery. Therefore, in order to secure the space for surgery, conventional surgery was performed such that a surgical assistant (e.g., a nurse) spread the incision site via direct pulling, or clamped the inside/outside of the incision site with a forceps, etc., connecting the wire to the forceps, and binding by pulling an instrument around the wire so as to wide open the incision site.

In the case of a conventional retractor which is to be inserted into the incision site, the structure and the shape of the retractor are complex, and the shape of the body of an upper part of the device is not maintained uniformly when the retractor is used by inserting into the skin incision area of a patient. Therefore, it is difficult to secure a sufficient view for surgery and removal of exudates, and thus, it is very important to maintain the shape of the body of an upper part of the retractor.

In addition, the spinal fixation screw operation in which the fixation screw is inserted into the spine of a patient, was conventionally performed such that the spinal lesion site of the patient was incised using a surgical instrument so as to ensure a view for the surgeon. With regard to the implantation of such a spinal fixation screw by such a general incision, there is an aspect that consistent safety in operation can be hardly secured due to surgical variables, such as excess discharge of blood and excess damage of soft tissue during the operation.

In a case where the operation is performed using a conventional retractor, not only the work of inserting the retractor into the incised skin is difficult, but also the expansion of the incision site by an operator is difficult. Therefore, there is required a very sophisticated level of skill and technique.

That is, the conventional retractor had inconveniencies in that it is difficult to use the retractor at the time of operation, and additionally, it is difficult to secure the view of the operator through the expansion of the incision site.

### PRIOR ART DOCUMENT

### Patent Document

(Patent Document 1) KR Patent No. 10-1172660 (Title of Invention: Wound Retractor For Laparoscopic Surgery, Published on Aug. 8, 2012)

(Patent Document 2) KR Patent No. 10-1817723 (Title of Invention: Surgical Wound Retractor, Published on Jan. 11, 2018. 01. 11)

### SUMMARY OF THE INVENTION

The present invention has been made in view of such circumstances, and an object of the present invention is to provide a retractor for spinal surgery, which allows an operator to obtain a maximum surgical view with a minimal incision by easily widening a skin incision site of a patient while minimizing the surgical incision area.

According to a preferred embodiment for achieving the above objects of the present invention, the retractor for spinal surgery according to the present invention may include a first retractor body, which has a semi-cylindrical shape and in which a space is formed to accommodate a plurality of sleeves, and simultaneously, a lower end thereof is inserted into a skin incision site; a second retractor body, which has the same shape as that of the first retractor body and is rotatably coupled to the first retractor body, and thereby accommodate the sleeves along with the first retractor body; and a pair of hinge units, which are mounted on an upper part of the first retractor body and the second retractor body, and in which an end of the first retractor body and an end of the second retractor body are hinged together by a connecting pin.

In the retractor for spinal surgery, both ends of the first retractor body and the second retractor body may be assembled through the hinge units in a superimposed manner.

The retractor for spinal surgery may further include a taper part, which is formed at a lower end of the first retractor body and the second retractor body and induces the insertion of the first retractor body and the second retractor body into the skin incision site.

The retractor for spinal surgery may further include a flange, which is coupled to the hinge units on an upper part of the first retractor body and the second retractor body, and simultaneously, at least one holding device is mounted thereon.

The retractor for spinal surgery may further include a pressing member, which are disposed in a direction perpendicular to the hinge units on an upper part of the flange to face with each other, and upon pressing the same, allowing the lower parts of the first retractor body and the second retractor body to become widely apart.

The pressing member may include a connecting groove which is formed on the surface of a plate and used for connecting an additional device thereto.

### BRIEF DESCRIPTION OF THE DRAWINGS

FIG. 1 is a diagram illustrating a retractor for spinal surgery according to an embodiment of the present invention.

FIG. 2 is a diagram illustrating the structure of an upper part of a retractor according to an embodiment of the present invention.

FIG. 3 is a diagram illustrating an assembled state of a sleeve according to an embodiment of the present invention.

FIG. 4 is a diagram illustrating a retractor and a sleeve according to an embodiment of the present invention.

FIG. 5 is a diagram illustrating the structure of a retractor according to an embodiment of the present invention.

FIG. 6 is a diagram illustrating a state in which a lower part of a retractor according to an embodiment of the present invention is expanded.

### DESCRIPTION OF EXEMPLARY EMBODIMENTS

Hereinafter, a retractor for spinal surgery according an embodiment of the present invention will be described with reference to the accompanying drawings. In particular, the present invention is not limited or restricted by the examples. Additionally, in describing the present invention, specific descriptions of known functions or constitutions may be omitted to clarify the gist of the present invention.

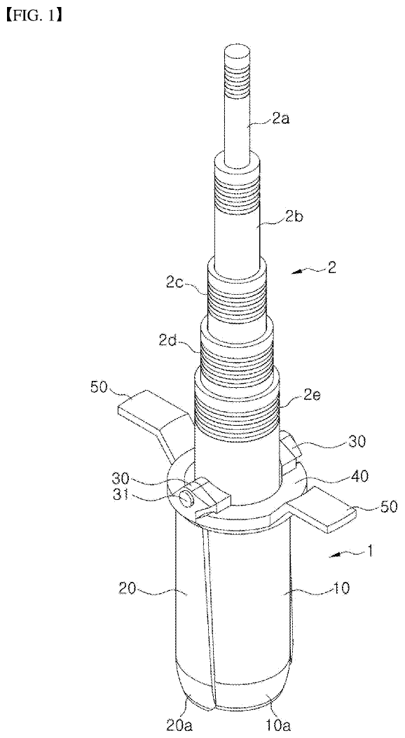

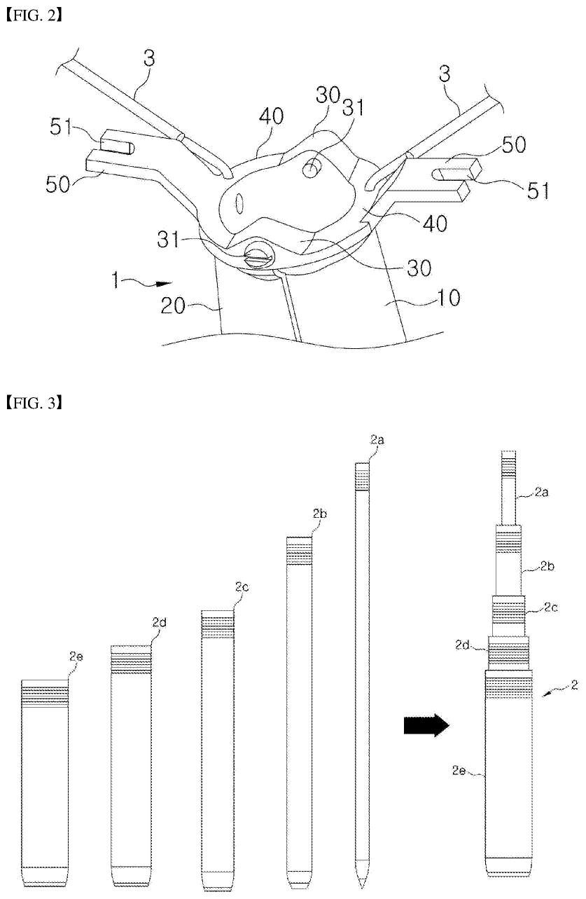

FIG. 1 is a diagram illustrating a retractor for spinal surgery according to an embodiment of the present invention. FIG. 2 is a diagram illustrating the structure of an upper part of a retractor according to an embodiment of the present invention.

Referring to FIGS. 1 and 2, a retractor for spinal surgery 1 may include a first retractor body 10, a second retractor body 20, and a hinge unit 30. After inserting the retractor 1 into a skin incision site of a patient, the retractor 1 may be widened so as to secure the view of the operator.

That is, in the retractor 1, a pair of bodies are connected through a hinge unit 30, and thus, the lower parts of the retractor 1 can be wide open by pushing an upper part of the bodies.

In this Example, a first retractor body 10 of the retractor 1 is one, in which a space is formed inside thereof so as to accommodate a plurality of sleeves 2, and simultaneously, a lower end thereof is inserted into a skin incision site, and establishes a semi-cylindrical shape such that a cylindrical member is cut in half.

The second retractor body 2, which has the same shape as that of the first retractor body 10, is rotatably coupled to the first retractor body 10, and thereby accommodates the sleeves 2 along with the first retractor body 10; and the retractor 1 establishes a semi-cylindrical shape by the coupling between the first retractor body 10 and the second retractor body 20.

In addition, the hinge units 30, which are mounted on an upper part of the first retractor body 10 and the second retractor body 20, respectively, and in which an end of the first retractor body 10 and an end of the second retractor body 20 are hinged together by a connecting pin 31, and the hinge units 30 are mounted at positions facing each other.

In this Example, the retractor 1 is established by the assembly of the first retractor body 10 and the second retractor body 20, and both ends of the first retractor body 10 and the second retractor body 20 may be assembled in a superimposed manner through the hinge units 30.

As described above, since the first retractor body 10 and the second retractor body 20 are assembled in a superimposed manner, no gap will be formed in the connecting part of the retractor 1 even if the lower parts of the retractor 1 are opened apart. As the overlapping area moves, the lower parts of the first retractor body 10 and the second retractor body 20 unfolded.

In addition, the retractor 1 may further include taper parts 10a and 20a, which are formed at a lower end of the first retractor body 10 and the second retractor body 20 and induce the insertion of the first retractor body 10 and the second retractor body 20 into the skin incision site.

The taper parts 10a and 20a can facilitate the insertion of the retractor 1 into the skin incision site. The taper parts 10a and 20a form an inwardly inclined shape at a lower end of the retractor 1 and thus allow the retractor 1 to be inserted more easily into the skin incision site of a patient.

In this Example, a flange 40 is mounted on an upper part of the retractor 1, and the flange 40 may be coupled to the hinge unit 30 on an upper part of the first retractor body 10 and the second retractor body 20, and simultaneously, at least one holding device 3 may be mounted thereon.

That is, after the retractor 1 is inserted into a skin incision site, the retractor 1 must be fixed to maintain the inserted state by connecting a plurality of holding devices 3 to a flange 40, which is formed on an upper part of the retractor 1.

Since it becomes difficult to perform the operation when the retractor 1 moves during the operation of a patient, the retractor 1 should be firmly fixed to the skin incision site using the holding device 3.

In addition, the retractor 1 fixed as described above must secure a view for the operator during operation by expanding the skin incision site. For this purpose, the retractor 1 may include pressing members 50, which are disposed in a direction perpendicular to the hinge unit 30 on an upper part of the flange 40 to face with each other, and which, upon pressing, allow the lower parts of the first retractor body 10 and the second retractor body 20 to become widely apart.

The structure of the retractor 1 is such that as the pressing members 50 provided on the upper part of the retractor 1 are pressed by a separate mechanism (not shown), the first retractor body 10 and the second retractor body 20, connected through the hinge unit 30, are opened while rotating about the hinge unit 30.

The pressing member 50 may include a connecting groove 51 which is formed on the surface of a plate and used for connecting an additional device thereto. The device can easily be mounted through the connecting groove 51, and the lower part of the retractor 1 can be expanded by pressing the pressing member 50.

FIG. 3 is a diagram illustrating an assembled state of a sleeve according to an embodiment of the present invention.

Referring to FIG. 3, the sleeve 2 is used to expand the diameter of a skin incision site before inserting the retractor 1 into the skin incision site. The sleeve 2 may include a first sleeve 2a with a pin shape, a second sleeve 2b to be inserted into the first sleeve 2a, a third sleeve 2c to be inserted into the second sleeve 2b, a fourth sleeve 2d to be inserted into the third sleeve 2c, and a fifth sleeve 2e to be inserted into the fourth sleeve 2d.

That is, the diameter of the second sleeve 2b is larger than that of the first sleeve 2a, the diameter of the third sleeve 2c is larger than that of the second sleeve 2b, the diameter of the fourth sleeve 2d is larger than that of the third sleeve 2c, and the diameter of the fifth sleeve 2e is larger than that of the fourth sleeve 2d. The second sleeve 2b to the fifth sleeve 2e are formed in a tubular shape, and thus, a sleeve with a larger diameter can be inserted into the outside of a sleeve with a smaller diameter.

Therefore, the first sleeve 2a is inserted into the skin of a first patient, the second sleeve 2b is inserted into the outside of the first sleeve 2a, the third sleeve 2c is inserted into the outside of the second sleeve 2b, the fourth sleeve 2d is inserted into the outside of the third sleeve 2c, and the fifth sleeve 2e is inserted into the outside of the fourth sleeve 2d. The diameter of the skin incision site of a patient becomes expanded by undergoing the above procedure.

After the retractor 1 is inserted into the outside of the fifth sleeve 2e, the first sleeve 2a through the fifth sleeve 2e are removed from the skin incision site of a patient. As a result, only the retractor 1 remains in the skin incision site of a patient.

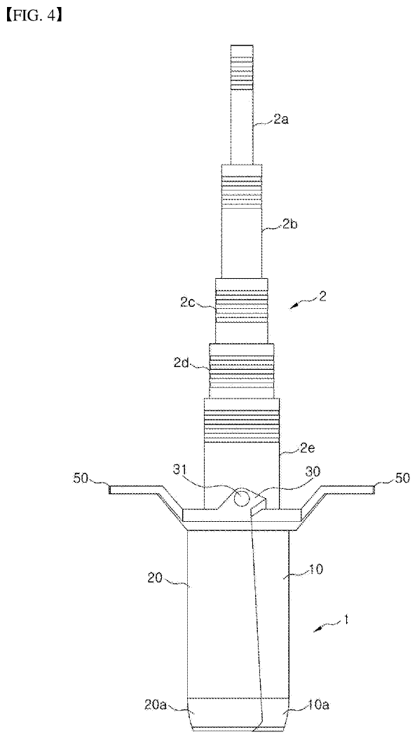

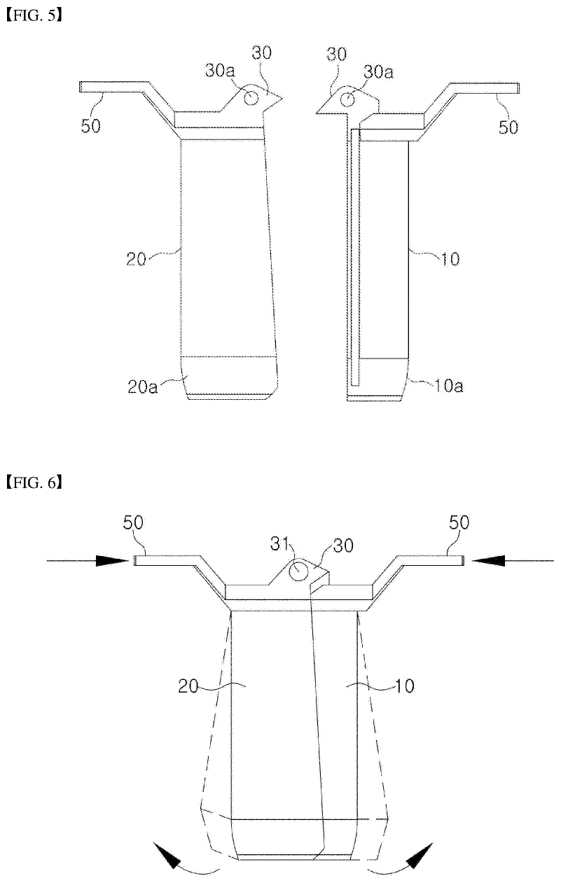

FIG. 4 is a diagram illustrating a retractor and a sleeve according to an embodiment of the present invention. FIG. 5 is a diagram illustrating the structure of a retractor according to an embodiment of the present invention. FIG. 6 is a diagram illustrating a state in which a lower part of a retractor according to an embodiment of the present invention is expanded.

Referring to FIGS. 4 to 6, the retractor 1 consists of a first retractor body 10, a second retractor body 20, and a hinge unit 30 connecting the first retractor body 10 and the second retractor body 20, and the retractor 1 accommodates a plurality of sleeves 2 inside of the retractor 1.

That is, a pin hole 30a is formed in the hinge unit 30, and the hinge unit 30 of the first retractor body 10 and the hinge unit 30 of the second retractor body 20 are connected with each other as a connecting pin 31 is inserted into the pin hole 30a. The first retractor body 10 and the second retractor body 20 can be rotatably assembled to each other through the hinge units 30.

The first retractor body 10 and the second retractor body 20 are rotated by pressing the pressing member 50 mounted on an upper part of the retractor 1, and thereby a lower part of the retractor 1 is expanded. In this state, the first sleeve 2a to the fifth sleeve 2e mounted on the inside of the retractor 1 are sequentially removed, and then the operation of the operator is performed through the retractor 1.

In particular, in the retractor 1, both ends of the first retractor body 10 and the second retractor body 20 are assembled through the hinge units 30 in a superimposed manner. As a result, no gap is formed in the connecting part of the retractor 1 even if the lower part of the retractor 1 is opened apart, and thus, it is possible to block the entry of foreign matters or blood into the retractor 1 through a gap during operation.

In the retractor 1, upon pressing the pressing member 50 on an upper part thereof, the area where the first retractor body 10 and the second retractor body 20 are overlapping moves and thereby lower parts of the first retractor body 10 and the second retractor body 20 are expanded, and accordingly, the skin incision site can be opened apart.

The operator proceeds with the operation in a state where the view is secured by widening the skin incision site through the retractor 1. During spinal surgery, the operator can safely and easily mount a spinal fixation screw onto the spinal area of a patient through the retractor.

Accordingly, a maximum surgical view for an operator can be secured with a minimal incision by easily widening a skin incision site of a patient while minimizing the surgical incision area through the retractor 1, and in addition, the skin incision site can be minimized and thus enabling a rapid recovery of the patient.

In addition, since the operation using the retractor 1 is convenient, the retractor 1 not only shortens the operation time, but also minimizes the pain of a patient.

In addition, since the retractor 1 can be more easily mounted on a skin incision site of a patient, the operation can be performed more accurately and rapidly.

While the present invention has been shown and described with reference to preferred embodiments for illustrating the principle of the present invention, the present invention is not to be construed as being limited to such constitutions and actions shown and described as such. It will be appreciated by those skilled in the art that numerous changes and modifications can be made to the present invention without departing from the spirit and scope of the appended claims.

[Explanation of Reference Numerals]

| | |
|---|---|
| 1: retractor | 2: sleeve |
| 2a: first sleeve | 2b: second sleeve |
| 2c: third sleeve | 2d: fourth sleeve |
| 2e: fifth sleeve | 3: holding device |
| 10: first retractor body | 10a: taper part |
| 20: second retractor body | 20a: taper part |
| 30: hinge unit | 30a: pin hole |
| 31: connecting pin | 40: flange |
| 50: pressing member | 51: connecting groove |

### Advantageous Effects

According to the retractor for spinal surgery according to the present invention, there is an advantage in that a maximum surgical view for an operator can be secured with a minimal incision by easily widening a skin incision site of a patient while minimizing the surgical incision area. In addition, the skin incision site can be minimized and thus the patient can recover rapidly.

In addition, since the operation using the retractor is convenient, not only the operation time can be shortened but also the pain after the operation can be minimized.

In addition, since the retractor can be mounted more easily at the skin incision site of a patient, the operation can be performed more accurately and rapidly.

What is claimed is:

1. A retractor for spinal surgery, comprising:
a first retractor body, which has a semi-cylindrical shape and in which a space is formed to accommodate a plurality of sleeves, and simultaneously, a lower end thereof is inserted into a skin incision site;
a second retractor body, which has the same shape as that of the first retractor body and is rotatably coupled to the first retractor body, and thereby accommodate the sleeves along with the first retractor body; and
a pair of hinge units, which are mounted on an upper part of the first retractor body and the second retractor body, and in which an end of the first retractor body and an end of the second retractor body are hinged together by a connecting pin.

2. The retractor for spinal surgery of claim 1, wherein both ends of the first retractor body and the second retractor body are assembled through the hinge units in a superimposed manner.

3. The retractor for spinal surgery of claim 1, further comprising a taper part, which is formed at a lower end of the first retractor body and the second retractor body and induces the insertion of the first retractor body and the second retractor body into the skin incision site.

4. The retractor for spinal surgery of claim 1, further comprising a flange, which is coupled to the hinge units on an upper part of the first retractor body and the second retractor body, and simultaneously, at least one holding device is mounted thereon.

5. The retractor for spinal surgery of claim 4, further comprising pressing members, which are disposed in a direction perpendicular to the hinge units on an upper part of the flange to face with each other, and which, upon pressing allow the lower parts of the first retractor body and the second retractor body to become widely apart.

6. The retractor for spinal surgery of claim 5, wherein the pressing member comprises a connecting groove which is formed on the surface of a plate and used for connecting an additional device thereto.
